(19) United States
(12) Patent Application Publication
(10) Pub. No.: US 2004/0204734 A1
(43) Pub. Date: Oct. 14, 2004

(54) Tunneling tool with subcutaneous transdermal illumination

(76) Inventors: Darrell Orvin Wagner, Isanti, MN (US); Jason Alan Shiroff, Shoreview, MN (US)

Correspondence Address: Crawford Maunu PLLC, 1270 Northland Drive, Suite 390, St. Paul, MN, 55120, US

(21) Appl. No.: 10/625,826

(22) Filed: Jul. 23, 2003

Related U.S. Application Data
(60) Provisional application No. 60/462,272, filed on Apr. 11, 2003.

(51) Int. Cl. A61B 17/00

(52) U.S. Cl. 606/190

(57) ABSTRACT

Subcutaneous tissue dissection tools, methods, systems, and kits incorporating transdermal illumination provide for enhanced navigation and depth determinations during subcutaneous tissue dissection. Subcutaneous dissection tools, methods, and systems provide access for deployment of subcutaneous electrodes, cans, and housings used in transthoracic defibrillation therapies, cardiac monitoring systems, transthoracic pacing therapies, or combinations of same. A dissection tool employing transdermal illumination includes a handle having a proximal end and a distal end. An elongated dissecting member extends from the distal end of the handle. A light source is provided within or to the dissection tool. The light source is adapted to provide a visible locating reference through the skin during subcutaneous tissue dissection.

### RELATED APPLICATIONS

This application claims the benefit of Provisional Patent Application Serial No. 60/462,272, filed on Apr. 11, 2003, to which priority is claimed pursuant to 35 U.S.C. §119(e) and which is hereby incorporated herein by reference.

### FIELD OF THE INVENTION

The present invention relates generally to tissue dissection instruments and, more particularly, to subcutaneous tissue dissection instruments and techniques incorporating a light source for transdermal illumination.

### BACKGROUND OF THE INVENTION

Implantable cardiac rhythm management systems have been used as an effective treatment for patients with serious arrhythmias. These systems typically include one or more leads and circuitry to sense signals from one or more interior and/or exterior surfaces of the heart. Such systems also include circuitry for generating electrical pulses that are applied to cardiac tissue at one or more interior and/or exterior surfaces of the heart. For example, leads extending into the patient's heart are connected to electrodes that contact the myocardium for sensing the heart's electrical signals and for delivering pulses to the heart in accordance with various therapies for treating arrythmias.

Implantable cardioverter/defibrillators (ICDS) have been used as an effective treatment for patients with serious cardiac arrhythmias. For example, a typical ICD includes one or more endocardial leads to which at least one defibrillation electrode is connected. Such ICDs are capable of delivering high-energy shocks to the heart, interrupting the ventricular tachyarrythmia or ventricular fibrillation, and allowing the heart to resume normal sinus rhythm. ICDs may also include pacing functionality.

Although ICDs are very effective at preventing Sudden Cardiac Death (SCD), most people at risk of SCD are not provided with implantable defibrillators. The primary reasons for this unfortunate reality include the limited number of physicians qualified to perform transvenous lead/electrode implantation, a limited number of surgical facilities adequately equipped to accommodate such cardiac procedures, and a limited number of the at-risk patient population that can safely undergo the required endocardial or epicardial lead/electrode implant procedure.

For reasons stated above, and for other reasons which will become apparent to those skilled in the art upon reading the present specification, there is a need for systems and methods that provide for sensing cardiac activity and delivering defibrillation and/or pacing therapies without the need for endocardial or epicardial leads/electrodes. There is a particular need for tools and techniques that facilitate implantation of such systems. The present invention fulfills these and other needs, and addresses deficiencies in known systems and techniques.

### SUMMARY OF THE INVENTION

The present invention is directed to subcutaneous dissection tools, methods and systems that, in general, provide access for deployment of subcutaneous electrodes, cans, and housings used in transthoracic defibrillation therapies, cardiac monitoring systems, transthoracic pacing therapies, or a combination of the above. Embodiments of the present invention include subcutaneous dissection tools, systems, and kits that include transdermal illumination during dissection.

According to one embodiment, a dissection tool of the present invention includes a handle having a proximal end and a distal end, and an elongated dissecting member having a proximal end and a distal end. The elongated dissecting member extends from the distal end of the handle and a light source is provided at the distal end of the dissecting member. The light source adapted to provide a visible locating reference through the skin.

The dissection tool may be straight or curved, rigid or malleable, and shaped to provide dissection paths suitable for the implantation of subcutaneous electrodes. A system incorporating dissection tools in accordance with the present invention may include a light source within the dissection tool, or may transmit light from an external source through the tool. The dissector may include a battery to power the light, and may have an On/Off switch located on the dissector or external to the dissector.

In further embodiments, the dissector includes a filter to filter the light, changing the lights' color or other optical property. A dissection system may also include a fluid delivery channel to deliver a pharmacological agent during dissection.

Another embodiment of the present invention is directed to a method of dissection. According to one approach, a method of dissecting subcutaneous tissue involves providing a dissection tool with a light source, dissecting subcutaneous tissue with the dissection tool, and transmitting light through the dermus during dissection. The dissection method may include steps of following the subcutaneous plane for dissection along the curvature of the rib cage, for example.

A further embodiment of the present invention provides methods of dissection using a curved or malleable transdermal illuminating dissector particularly suited to dissect a path for subcutaneous electrode placement. Yet another embodiment of the present invention is directed to kits that include selected tools, implements, and transdermal illuminating devices for performing subcutaneous dissection including fluid delivery.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIGS. 1A and 1B are views of a transthoracic cardiac monitoring and/or stimulation device as implanted in a patient;

FIG. 2 is a plan view of a subcutaneous dissection system in accordance with the present invention;

FIG. 3 illustrates a method of dissection using transdermal illumination;

FIGS. 4A and 4B illustrate light sources in accordance with two embodiments of the present invention;

FIGS. 5A and 5B are plan views of two embodiments of dissectors in accordance with the present invention;

FIGS. 6A, 6B and 6C are plan views of further embodiments of dissectors in accordance with the present invention; and

FIG. 7 is a magnified sectional view of the distal end of a dissector that incorporates both transdermal illumination and fluid delivery in accordance with an embodiment of the present invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings, which form a part hereof, and in which is shown by way of illustration various embodiments in which the invention may be practiced. It is to be understood that other embodiments may be utilized, and structural and functional changes may be made without departing from the scope of the present invention.

A device in accordance with the present invention can include one or more of the features, structures, methods, or combinations thereof described herein below. For example, a subcutaneous dissector or dissection method can be implemented to include one or more of the advantageous features and/or processes described below. It is intended that such a dissection device or method need not include all of the features and functions described herein, but can be implemented to include selected features and functions that provide for unique structures and/or functionality.

In general terms, a dissection tool of the present invention can be used to facilitate implantation of a subcutaneous cardiac monitoring and/or stimulation device. One such device is an implantable transthoracic cardiac sensing and/or stimulation (ITCS) device that can be implanted under the skin in the chest region of a patient. The ITCS device may, for example, be implanted subcutaneously such that all or selected elements of the device are positioned on the patient's front, back, side, or other body locations suitable for sensing cardiac activity and delivering cardiac stimulation therapy. It is understood that elements of the ITCS device may be located at several different body locations, such as in the chest, abdominal, or subclavian region with electrode elements respectively positioned at different regions near, around, in, or on the heart. A dissection tool and methodology of the present invention can be used to provide electrode and device access at various subcutaneous body locations.

The primary housing (e.g., the active or non-active can) of the ITCS device, for example, can be configured for positioning outside of the rib cage at an intercostal or subcostal location, within the abdomen, or in the upper chest region (e.g., subclavian location, such as above the third rib). In one implementation, one or more electrodes can be located on the primary housing and/or at other locations about, but not in direct contact with the heart, great vessel or coronary vasculature. In another implementation, one or more electrodes can be located in direct contact with the heart, great vessel or coronary vasculature, such as via one or more leads implanted by use of conventional transvenous delivery approaches. In another implementation, for example, one or more subcutaneous electrode subsystems or electrode arrays can be used to sense cardiac activity and deliver cardiac stimulation energy in an ITCS device configuration employing an active can or a configuration employing a non-active can. Electrodes can be situated at anterior and/or posterior locations relative to the heart.

Due to the number of combinations of electrodes and ITCS devices, and the variability of anatomy and the presentation of conditions amongst patients, surgical kits are often assembled prior to surgery to provide the basic combinations of devices, leads, and ancillary components necessary to perform the surgical procedure. As will be discussed in detail below, dissection kits of the present invention can be assembled to include one or more dissection tools, including those that provide for transdermal illumination with or without fluid delivery, one or more electrodes and leads, one or more cans or housings, and combinations of these and other subcutaneous components.

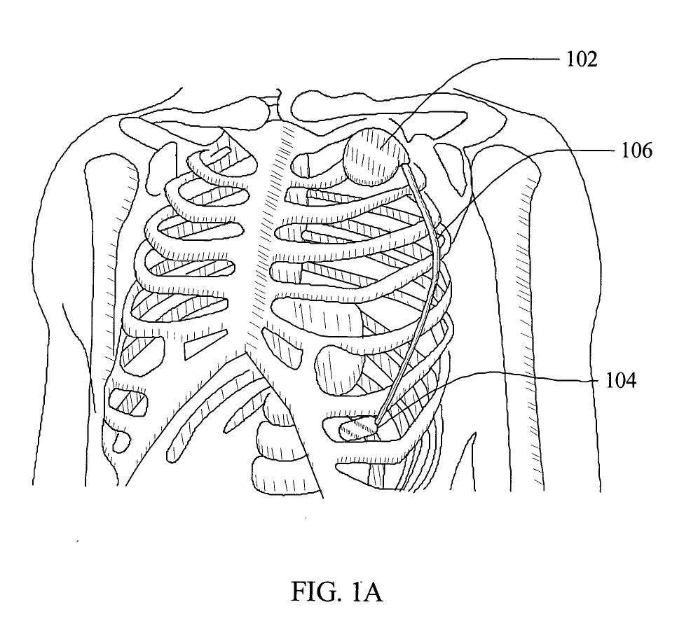

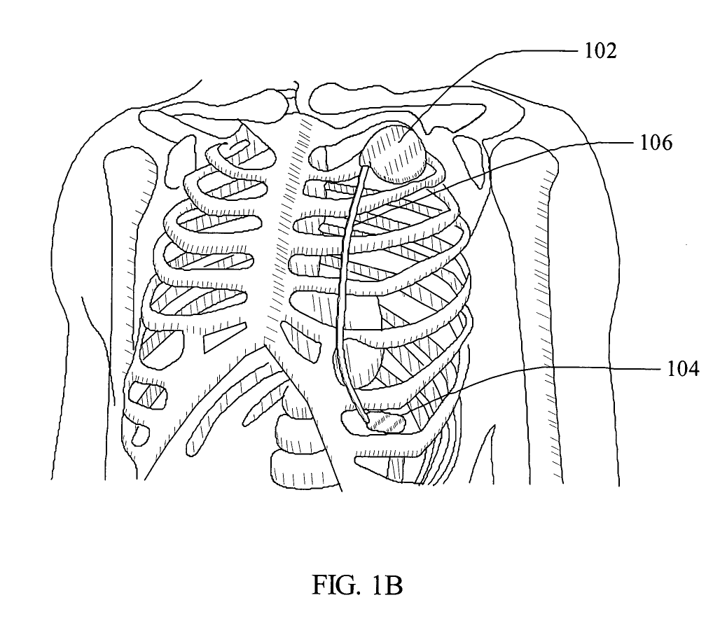

Referring now to FIGS. 1A and 1B of the drawings, there is shown a configuration of a transthoracic cardiac sensing and/or stimulation (ITCS) device implanted in the chest region of a patient at different locations by use of a dissection tool of the present invention. In the particular configuration shown in FIGS. 1A and 1B, the ITCS device includes a housing 102 within which various cardiac sensing, detection, processing, and energy delivery circuitry can be housed. The housing 102 is typically configured to include one or more electrodes (e.g., can electrode and/or indifferent electrode). Although the housing 102 is typically configured as an active can, it is appreciated that a non-active can configuration may be implemented, in which case at least two electrodes spaced apart from the housing 102 are employed. An ITCS system according to this approach is distinct from conventional approaches in that it is preferably configured to include a combination of two or more electrode subsystems that are implanted subcutaneously in the anterior thorax.

In the configuration shown in FIGS. 1A and 1B, a subcutaneous electrode 104 can be positioned under the skin in the chest region and situated distal from the housing 102. The subcutaneous and, if applicable, housing electrode(s) can be positioned about the heart at various locations and orientations, such as at various anterior and/or posterior locations relative to the heart. The subcutaneous electrode 104 is electrically coupled to circuitry within the housing 102 via a lead assembly 106. One or more conductors (e.g., coils or cables) are provided within the lead assembly 106 and electrically couple the subcutaneous electrode 104 with circuitry in the housing 102. One or more sense, sense/pace or defibrillation electrodes can be situated on the elongated structure of the electrode support, the housing 102, and/or the distal electrode assembly (shown as subcutaneous electrode 104 in the configuration shown in FIGS. 1A and 1B).

In one configuration, the lead assembly 106 is generally flexible and has a construction similar to conventional implantable, medical electrical leads (e.g., defibrillation leads or combined defibrillation/pacing leads). In another configuration, the lead assembly 106 is constructed to be somewhat flexible, yet has an elastic, spring, or mechanical memory that retains a desired configuration after being shaped or manipulated by a clinician. For example, the lead assembly 106 can incorporate a gooseneck or braid system that can be distorted under manual force to take on a desired shape. In this manner, the lead assembly 106 can be shape-fit to accommodate the unique anatomical configuration of a given patient, and generally retains a customized shape after implantation. Shaping of the lead assembly 106 according to this configuration can occur prior to, and during, ITCS device implantation.

In accordance with a further configuration, the lead assembly 106 includes a rigid electrode support assembly, such as a rigid elongated structure that positionally stabilizes the subcutaneous electrode 104 with respect to the housing 102. In this configuration, the rigidity of the elongated structure maintains a desired spacing between the subcutaneous electrode 104 and the housing 102, and a desired orientation of the subcutaneous electrodes 104/housing 102 relative to the patient's heart. The elongated structure can be formed from a structural plastic, composite or metallic material, and comprises, or is covered by, a biocompatible material. Appropriate electrical isolation between the housing 102 and the subcutaneous electrode 104 is provided in cases where the elongated structure is formed from an electrically conductive material, such as metal.

In one configuration, the rigid electrode support assembly and the housing 102 define a unitary structure (i.e., a single housing/unit). The electronic components and electrode conductors/connectors are disposed within or on the unitary ITCS device housing/electrode support assembly. At least two electrodes are supported on the unitary structure near opposing ends of the housing/electrode support assembly. The unitary structure can have an arcuate or angled shape, for example.

According to another configuration, the rigid electrode support assembly defines a physically separable unit relative to the housing 102. The rigid electrode support assembly includes mechanical and electrical couplings that facilitate mating engagement with corresponding mechanical and electrical couplings of the housing 102. For example, a header block arrangement can be configured to include both electrical and mechanical couplings that provide for mechanical and electrical connections between the rigid electrode support assembly and housing 102. The header block arrangement can be provided on the housing 102 or the rigid electrode support assembly. Alternatively, a mechanical/electrical coupler can be used to establish mechanical and electrical connections between the rigid electrode support assembly and the housing 102. In such a configuration, a variety of different electrode support assemblies of varying shapes, sizes, and electrode configurations can be made available for physically and electrically connecting to a standard ITCS device.

Depending on the configuration of a particular ITCS device, a delivery system incorporating transdermal illumination according to the present invention can advantageously be used to facilitate proper placement and orientation of the ITCS device housing and subcutaneous electrode(s). For example, when a clinician is performing dissection to create access for lead placement, conventional tunneling tools may be used to tunnel subcutaneously prior to lead placement. Conventional navigation for lead placement typically involves use of palpitation in the region around the distal end of the tool to try to determine the location of the most distal portion. Intervening tissues and structures can interfere with the clinician's perception of the location of this distal end, causing extended time for surgical procedures or possibly non-optimal electrode placement.

A dissector according to the present invention provides a light source that projects light from the distal end of the tunneling tool for improved navigation and placement of subcutaneous leads. While dissecting with the illuminating tunneling tool subcutaneously, light from the distal end of the tool serves as a visual aid to identify the location of the distal end along the dissection path. The light emanating from the tool is transmitted through the tissue and skin and is readily visible by the clinician. The relative level of light perceived by the clinician can also serve to indicate the depth of the dissection tool's distal end within the subcutaneous tissue.

An illuminating tunneling tool of the present invention advantageously enables medical professionals to place leads, cans, and other components subcutaneously with more accuracy, at the desired depth. In one configuration of a dissecting tool in accordance with the present invention, a long metal rod similar to conventional trocars, but including transdermal illumination, can be used to perform small diameter blunt tissue dissection of the subdermal layers. This tool may be pre-formed to assume a straight or curved shape to facilitate placement of the subcutaneous electrode, or may be malleable to bend to a desired shape determined by the clinician.

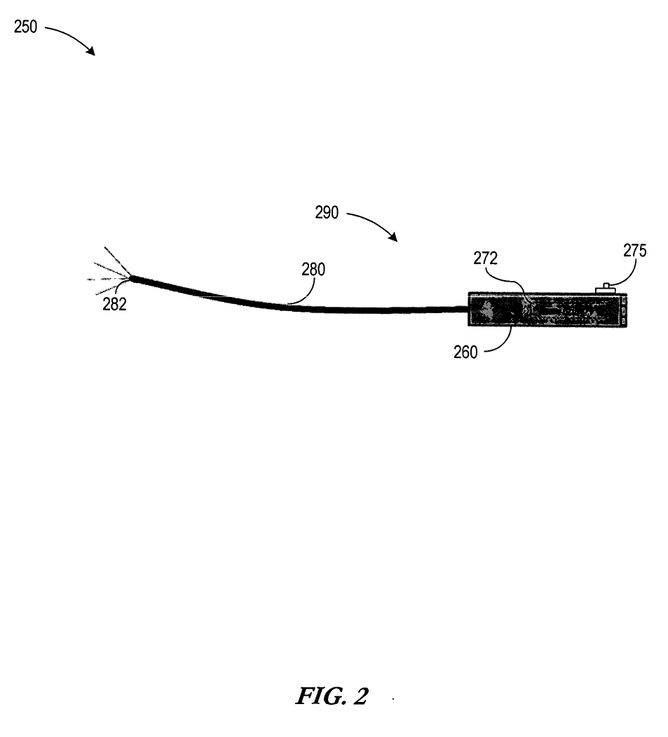

Referring now to FIG. 2, one embodiment of a curved dissecting tool according to the present invention is illustrated. A transdermally illuminating (TI) dissection system 250 is shown, including an internally powered TI dissector 290. The internally powered TI dissector 290 includes a handle 260 containing a power source 272. A light source 282 emits light at or near the distal end of an elongated dissecting member 280. The light emanating from the distal end of the dissecting member 280 can be used to illuminate a path of dissection, such as for purposes of transdermally guiding the dissector 290. A switch 275 controls the emission of light from a light source 282, such as by turning the light source 282 on and off. The switch 275 or other switch can also be used to vary the intensity of the light emitted by the light source 282.

A non-exhaustive, non-limiting list of light emitting devices for the light source 282 includes, for example, an incandescent bulb, a light emitting diode (LED), a florescent light source, a vapor lamp, an arc lamp, a plasma light source and a halogen bulb. The light source 282 may be toggled on and off via a switch 275. The switch 275 is illustrated on the handle 260, but may be located internally or externally to the TI dissector 290. For example, the switch 275 may be simply a pull-tab between two contacts that is pulled to initiate power to the light source 282 until the power is exhausted. The switch may be a physical switch, or may be a computer controlled switch such as, for example, a voice-activated relay. In another embodiment, the switch 275 may be located on an external light source, where the light is transmitted to the TI dissector 290 via an optical transmission arrangement, as will be described more fully below.

A non-exhaustive, non-limiting list of power sources 272 includes, for example, a storage battery, a fuel cell, a rechargeable battery, an electrochemical cell or other suitable power source located within the TI dissector 290. The power source for the dissector 290 may also be an external source. For example, the power source 272 may simply be an electrically isolated source that obtains power from a standard wall outlet (110 or 220 volt, for example). Electrically isolated power is coupled to the TI dissector 290 by a power cord.

In FIG. 2, the elongated dissecting member 280 is illustrated as a slightly curved member. However, it is contemplated that the elongated dissection member 280 may have any useful shape. For example, the elongated dissecting member 280 may be curved in one or more planes. The elongated dissecting member 280 may be pre-formed in a curved shape, or may be malleable into any shape desired by the clinician.

The elongated dissecting member 280 may, for example, have a pre-defined curvature to properly position an ITCS electrode relative to the can for proper location of the electric field relative to a patients' heart. The elongated dissecting member 280 may also, or alternately, have a pre-defined curvature that can easily follow the curvature of the rib cage for proper dissection. It is contemplated that any combination of predefined shapes with varying levels of malleability can be utilized in the present invention.

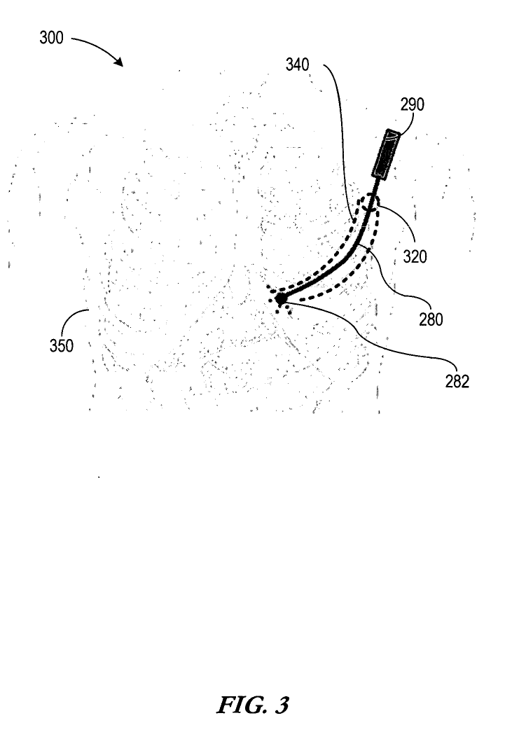

FIG. 3 illustrates a method of dissection 300 using transdermal illumination consistent with ITCS placement as illustrated in FIG. 1A. The TI dissector 290 may be placed into subcutaneous tissue through an initial incision in the dermus at an entry point 320 of a torso 350. With the light source 282 on, a transdermally illuminated spot will appear at a location along the thorax of the torso 350 consistent with the location of the light source emission. For example, if light is emitted from the light source 282 at the distal end of the elongated dissecting member 280, the clinician will discern the location of the distal end of the TI dissector 290 by observing where the light appears through the dermus.

By observing the relative quality of the light, the clinician can optimally direct the dissection path so that placement of subcutaneous electrodes is optimized. For example, by observing the intensity, color, and/or size of the spot illuminated through the dermus, the clinician could discern depth of dissection, location of dissection, and intervening structures between the dissection path and the surface of the skin, and dissect along an optimal path 340.

The clinician may then either place subcutaneous electrodes into the dissected path, or continue dissection crainially and medially from the entry point 320 to provide for placement of the can. Therefore, a method in accordance with the present invention may involve: providing a dissection tool with a transdermal illumination source; dissecting subcutaneous tissue with the dissection tool; and transdermally illuminating a path of dissection using light from the transdermal illumination source. The clinician may further proceed to guide the dissection using the light source, and may also perform other steps such as, for example, delivering a pharmacological agent along the path of dissection.

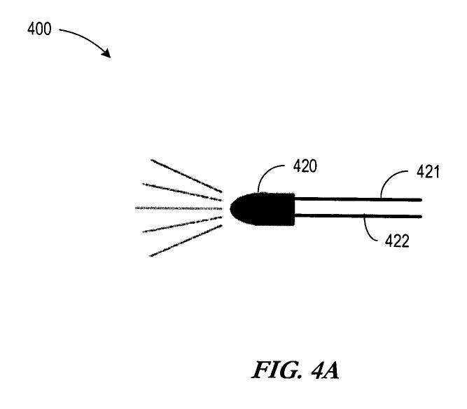

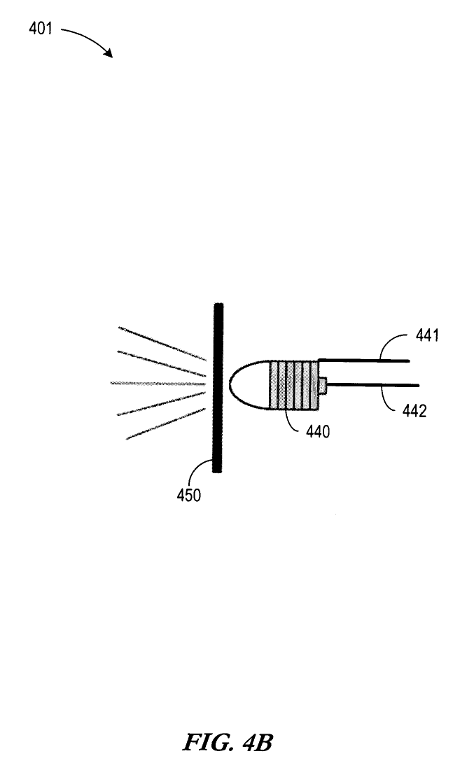

Referring now to FIGS. 4A and 4B, two light-emitting arrangements 400 and 401 are respectively illustrated as possible implementations of the light source 282 shown in FIGS. 2 and 3. In FIG. 4A, an LED 420 is shown connected to two conductors, a positive wire 422 and a negative wire 421. Wires 421 and 422 are connectable to a power source (not shown). The LED 420 may be a colored LED, a white-light LED, or other solid-state light-emitting device.

Referring to FIG. 4B, an incandescent bulb 440 having a positive wire 442 and a negative wire 441 may be used as the light source 282 shown in FIGS. 2 and 3. Wires 441 and 442 are connectable to a power source (not shown). The incandescent bulb 440 may be a standard filament bulb, or other incandescent light source.

It may be desirable to alter the color of the light by placement of a filter 450 on or at the light source, or in the path of the light as is illustrated by the filter 450 in front of the incandescent bulb 440. By altering the quality of the color from the light source, the clinician may better appreciate and discern the depth of the dissection and intervening tissue types such as vasculature, nerve bundles, muscles, or other tissues of interest.

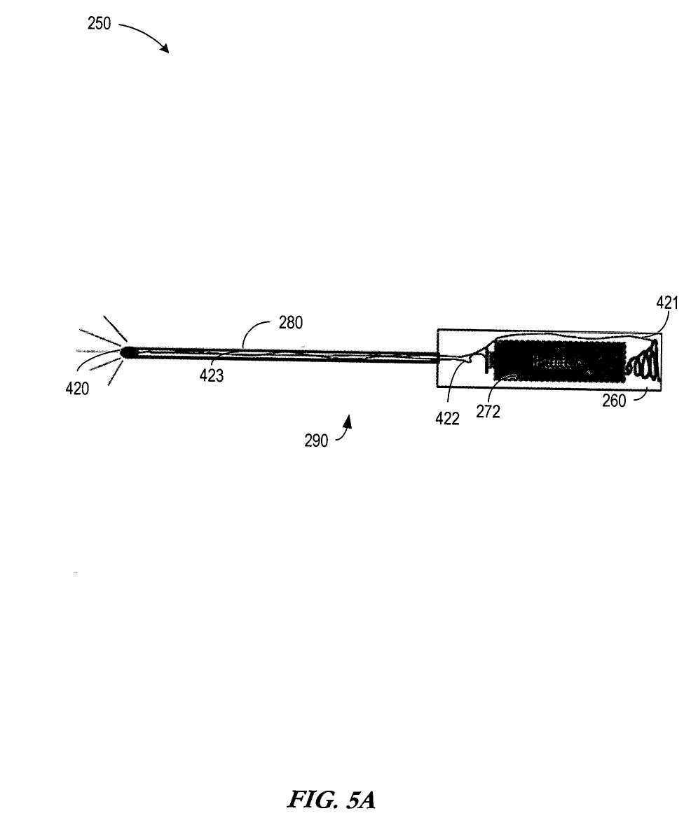

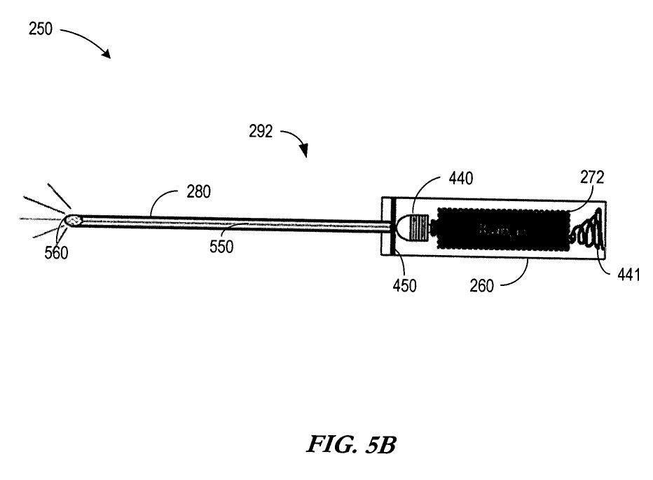

FIGS. 5A and 5B illustrate two embodiments of the TI dissection system 250 in accordance with the present invention. In FIG. 5A, the TI dissector 290 is shown having the LED 420 provided at the distal end of the elongated dissecting member 280, with a wire set 423 electrically connecting the LED 420 to the power source 272.

In FIG. 5B, a TI dissector 292 is shown having the incandescent bulb 440 in the handle 260, where light can be filtered through the optional filter 450 and transmitted through a light pipe 550 to a light exit 560. The light exit 560 may be at the distal end of dissecting member 280 as illustrated, or may be located at one or a plurality of locations along the dissecting member 280.

The light pipe 550 may be, for example, an acrylic rod, an optical fiber, a fiber optic bundle, a quartz rod, or any other suitable light transmission medium. The filter 450 may be permanently rigidly placed, or be removable or adjustable in color or other light transmission properties. The filter may be, for example, an acetate sheet, colored glass, a partially reflecting mirror, a polarizing lens, colored plastic, or other suitable material.

As illustrated in FIG. 5B, wiring for the light source, in this case incandescent bulb 440, may be internal to the structure of the device. In the example shown for the TI dissector 292, the negative wire 441 has been partially replaced by an electrical connection defined between an electrically conductive portion or element of the handle 260 and the bulb 440, as is employed in flashlights known in the art. The positive wire has been replaced by direct contact of the bulb 440 with the positive terminal of the power source 272, here illustrated as a battery.

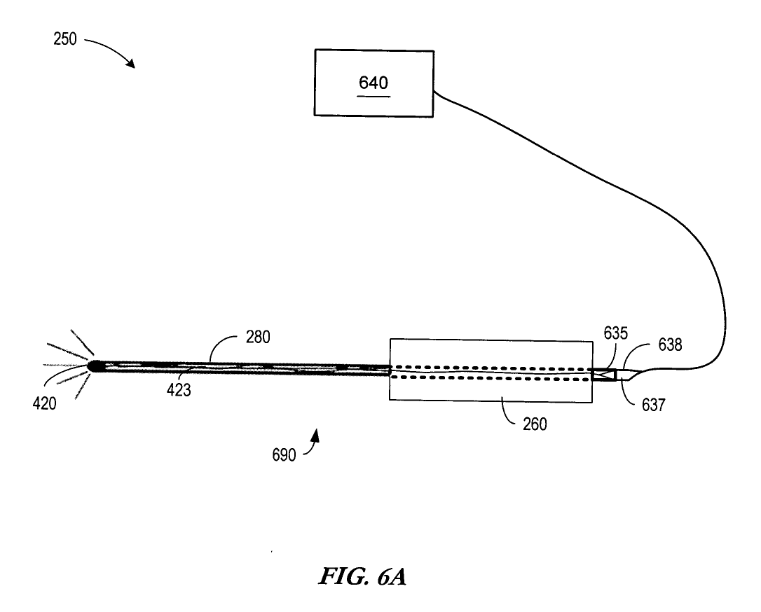

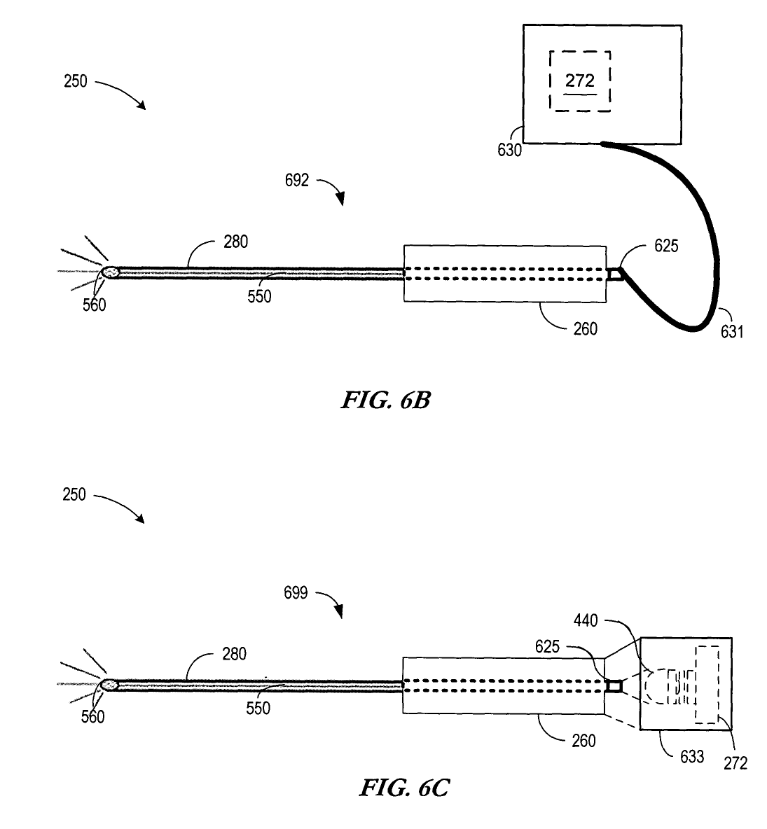

Now referring to FIGS. 6A, 6B and 6C, other embodiments are illustrated. Referring to FIG. 6A, a TI dissector 690 has a wire set 423 electrically connecting the LED 420 to an electrical connector 635. The connector 635 has a first pin 637 and a second pin 638 to mate with an external power source 640. The connector 635 is shown directly outside of the handle 260, for example mounted on or integrated into the handle 260, but may extend on a wire cable as far as desired for ease of use and connectivity to the power source 640.

Referring to FIG. 6B, a TI dissector 692 is shown with the light pipe 550 extending through the handle 260 to an optical connector 625. The optical connector 625 may be connected by a fiber optic cable 631 or other light transmission system to provide externally generated light into the TI dissector 692. For example, the optical connector 625 may be adapted to connect and/or mate with light sources available in the operating room that are normally used to illuminate through an endoscope for laparoscopic surgery, for example. In the illustrative embodiment of FIG. 6B, an external light generator 630, which incorporates a power supply 272, produces light which is optically coupled to the distal end of the TI dissector 692 via fiber optic cable 631, optical connector 625, and light pipe 550.

Referring to FIG. 6C, a TI dissector 699 is shown with the light pipe 550 extending through the handle 260 to an optical connector 625. The optical connector 625 may be connected directly to an external source 633 to provide light into the TI dissector 699. For example, the optical connector 625 may be adapted to connect and/or mate with light sources such as, for example, a flashlight. The TI dissector 699 can incorporate an internal battery 272 or connect to an external power supply (not shown).

In accordance with another embodiment, an ITCS device delivery tool of the present invention can incorporate a fluid delivery system in addition to a transdermal illumination system. The fluid delivery system can be used to communicate various fluids, such as pharmacological agents and irrigation fluids, to tissue subject to dissection. For example, a TI dissector can be configured to include a handle having a proximal end and a distal end, and an elongated dissecting member having a proximal end and a distal end. The elongated dissecting member extends from the distal end of the handle. A fluid channel system extends from at least the proximal end of the elongated dissecting member to the distal end of the elongated dissecting member.

The fluid channel system terminates in a port system. The port system may include one or more apertures, one or more channels, and be adapted to transport fluids such as, for example, irrigation fluids, fluids having analgesics, antibiotics, hemostatic agents, healing accelerating agents, agents that improve the electrical properties of tissue, and combinations of fluids and agents. In alternate embodiments, the apertures of the port system may have associated valves or covers such as, for example, flapper valves to keep debris out of the fluid channels. A system incorporating a dissection tool according to this embodiment may include fluid storage, a pump, and tubing for fluid delivery.

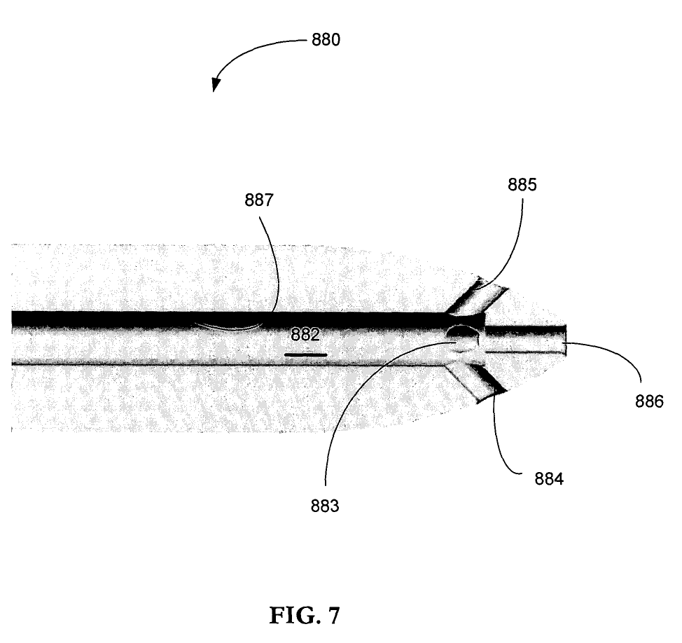

FIG. 7 is a magnified sectional view of the distal end of a dissector that incorporates both transdermal illumination and a fluid delivery system. In FIG. 7, the TI dissector includes an elongated dissecting member 880 having an illumination lumen 886 and a fluid delivery lumen 882. As shown, the illumination lumen 886 resides within, but is separated from, the fluid delivery lumen 882. In this arrangement, the respective diameters of the illumination and fluid delivery lumens are dimensioned to provide a longitudinal gap which defines an axial channel 887 within which fluids can be transported. The illumination lumen 886 can be configured to accommodate components associated with the various illumination embodiments described above. For example, the illumination lumen 886 can be a light tube or can house an illumination source, electrical wires, and/or a fiber-optic cable.

In another configuration, two, three or more separate lumens can be provided within the dissecting member 880. At least one of the lumens can be used as an illumination lumen as described immediately above. One or more other lumens can be provided for fluid delivery. For example, a single fluid delivery lumen can be provided to deliver a pharmacological agent or an irrigation fluid. By way of further example, two independent fluid delivery lumens can be provided for delivering particular fluids in each of the two lumens (e.g., a pharmacological agent delivered in one lumen, and an irrigation fluid delivered in the second lumen).

In the embodiment of FIG. 7, there is shown a port system which includes an axial channel 887 and a number of lateral apertures 883, 884, and 885. Depiction of the apertures 883, 884, and 885 is for purposes of clarity of explanation, and not of limitation. It is contemplated that a single aperture, or any number of apertures, may be located on the elongated dissecting element 880 at any location for dispensing a fluid from the TI dissector 880.

For example, a single or series of apertures may be located proximally from the distal end of the elongated dissecting member 880 to provide a pharmacological agent or other fluid anywhere along the path of dissection. If, for example, an analgesic is delivered during dissection, it may be efficacious to provide a number of ports of port system at the distal end of the dissector to ease the pain of dissection, but also to deliver incremental amounts of analgesic along the length of the elongated dissecting member 880 as the dissector progresses into tissue.

A pharmacological agent may be delivered continuously from the port system during dissection. It is also contemplated that the pharmacological agent may be delivered in bolus fashion at time intervals, or only delivered on demand through actuation of a fluid control. For example, the pharmacological agent may be delivered when a clinician desires to flush out debris from the dissection path, and may deliver saline solution to remove the debris.

Exemplary delivery tools, aspects of which can be incorporated into an ITCS device delivery tool in accordance with the present invention, are disclosed in commonly owned U.S. Pat. No. 5,300,106 and U.S. patent application entitled “Subcutaneous Dissection Tool Incorporating Pharmacological Agent Delivery,” filed concurrently herewith under Attorney Docket No. GUID.614PA, which are hereby incorporated herein by reference. These and other conventional delivery devices can advantageously be modified to incorporate a transdermal illumination capability and other structural and functional features as described herein.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

What is claimed is:

1. A dissection tool, comprising:
a handle having a proximal end and a distal end;
an elongated dissecting member having a proximal end and a distal end, the elongated dissecting member extending from the distal end of the handle; and
a light source provided at the distal end of the dissecting member, the light source adapted to provide a visible locating reference through the skin.

2. The dissection tool of claim 1, further comprising a battery adapted to provide power to the light source.

3. The dissection tool of claim 1, further comprising a power line having a distal end extending from the light source and a proximal end extending to at least a surface of the handle, the proximal end of the power line coupled to a connector adapted to matingly engage a connector of an external power source.

4. The dissection tool of claim 1, further comprising a switch provided on the handle of the dissection tool, wherein the switch is adapted to toggle the light source off and on.

5. The dissection tool of claim 1, wherein the light source comprises a light emitting diode.

6. The dissection tool of claim 1, wherein the light source comprises an incandescent bulb.

7. The dissection tool of claim 1, wherein the light source comprises a color filter.

8. The dissecting tool of claim 1, further comprising a fluid channel system extending from the proximal end of the elongated dissecting member to the distal end of the elongated dissecting member, the fluid channel system terminating in a port system.

9. The dissecting tool of claim 8, wherein the fluid channel system is adapted to transport a pharmacological agent.

10. The dissecting tool of claim 9, wherein the pharmacological agent comprises one or more of an analgesic, an antibiotic, and an antiseptic agent.

11. The dissecting tool of claim 8, wherein a first fluid channel is adapted to transport irrigation fluid and a second fluid channel is adapted to transport a pharmacological agent.

12. A dissection tool, comprising:
a handle having a proximal end and a distal end;
an elongated dissecting member having a proximal end and a distal end, the dissecting member extending from the distal end of the handle;
a light source provided proximally of the distal end of the dissecting member; and
a transmission member adapted to couple light from the light source to the distal end of the dissecting member, wherein light projected from the distal end of the dissecting member provides a visible locating reference through the skin.

13. The dissection tool of claim 12, wherein the light source is positioned at the handle.

14. The dissection tool of claim 12, wherein the light source is located externally of the dissection tool.

15. The dissection tool of claim 12, wherein the light source is located externally of the dissection tool, and the transmission member extends from the distal end of the dissecting member to at least a surface of the handle, a proximal end of the transmission member coupled to a connector adapted to matingly engage a connector of the externally located light source.

16. The dissection tool of claim 15, wherein the transmission member comprises a fiber-optic cable.

17. The dissection tool of claim 12, wherein the transmission member comprises a light pipe.

18. The dissection tool of claim 12, wherein the transmission member comprises a fiber-optic cable.

19. The dissection tool of claim 12, wherein the light source comprises a light emitting diode.

20. The dissection tool of claim 12, wherein the light source comprises an incandescent bulb.

21. The dissection tool of claim 12, wherein the light source comprises a color filter.

22. The dissection tool of claim 12, further comprising a battery adapted to provide power to the light source.

23. The dissection tool of claim 12, further comprising a power line having a distal end extending from the light source and a proximal end extending to at least an exterior surface of the handle, the proximal end of the power line coupled to a connector adapted to matingly engage a connector of an external power source.

24. The dissection tool of claim 12, further comprising a switch, wherein the switch is adapted to toggle the light source between off and on states.

25. The dissecting tool of claim 12, further comprising a fluid channel system extending from the proximal end of the elongated dissecting member to the distal end of the elongated dissecting member, the fluid channel system terminating in one or more ports.

26. The dissecting tool of claim 25, wherein the fluid channel system is adapted to transport a pharmacological agent.

27. The dissecting tool of claim 26, wherein the pharmacological agent comprises one or more of an analgesic, an antibiotic, and an antiseptic agent.

28. The dissecting tool of claim 25, wherein a first fluid channel is adapted to transport irrigation fluid and a second fluid channel is adapted to transport a pharmacological agent.

29. A method of dissecting subcutaneous tissue, comprising:
providing a dissection tool with a transdermal illumination source;
dissecting subcutaneous tissue with the dissection tool; and
transdermaiiy illuminating a path of dissection using light from the transdermal illumination source.

30. The method of claim 29, further comprising coupling light from an external light source to the transdermal illumination source provided at a distal end of the dissection tool.

31. The method of claim 29, further comprising coupling internally generated light to the transdermal illumination source provided at a distal end of the dissection tool.

33. The method of claim 29, further comprising coupling power from a power source external to the dissection tool to the transdermal illumination source.

34. The method of claim 29, further comprising coupling power from a power source internal to the dissection tool to the transdermal illumination source.

35. The method of claim 29, wherein the transdermal illumination source comprises a light emitting diode.

36. The method of claim 29, wherein the transdermal illumination source comprises an incandescent bulb.

37. The method of claim 29, further comprising filtering the light to achieve a desired color.

38. The method of claim 29, wherein dissection is performed only in a subcutaneous tissue plane.

39. The method of claim 29, further comprising guiding the dissection using the transdermal illumination.

40. The method of claim 29, further comprising delivering a fluid along the path of dissection from the dissection tool.

41. The method of claim 40, wherein the fluid comprises a pharmacological agent.

42. The method of claim 41, wherein the pharmacological agent comprises one or more of an analgesic, an antibiotic, and an antiseptic agent.

43. The method of claim 29, further comprising delivering a pharmacological fluid and an irrigation fluid along the path of dissection from the dissection tool.

44. A dissection tool, comprising:
a handle having a proximal end and a distal end;
an elongated dissecting member extending from the distal end of the handle; and
means for illuminating a path of subcutaneous tissue dissection.

45. The dissection tool of claim 44, further comprising means for coupling external power to the illuminating means.

46. The dissection tool of claim 44, further comprising means for providing internal power to the illuminating means.

47. The dissection tool of claim 44, further comprising means for switching the illuminating means between off and on states.

48. The dissection tool of claim 44, further comprising means for coupling light from an external light source to the illuminating means.

49. The dissection tool of claim 44, further comprising means for coupling light from an internal light source to the illuminating means.

50. The dissection tool of claim 44, wherein the illuminating means comprises a light emitting diode.

51. The dissection tool of claim 44, wherein the illuminating means comprises an incandescent bulb.

52. The dissection tool of claim 44, wherein the illuminating means comprises means for color filtering light.

53. A dissection tool, comprising:
a handle having a proximal end and a distal end;
an elongated dissecting member extending from the distal end of the handle, the dissecting member having a proximal end, a distal end, and at least one curved portion; and
an optical location indicator provided at the distal end of the dissecting member and adapted to provide a visual indication of a location of the distal end of the dissecting member through the dermus.

54. The dissection tool of claim 53, wherein the elongated dissecting member has a curvature appropriate for dissection along a plane that follows a curvature of a rib-cage.

55. The dissection tool of claim 53, wherein the elongated dissecting member has a generally arcuate shape.

56. The dissection tool of claim 53, further comprising means for providing power to the optical location indicator.

57. The dissection tool of claim 53, further comprising means for switching the optical location indicator between off and on states.

58. The dissection tool of claim 53, wherein the optical location indicator comprises a light emitting diode.

59. The dissection tool of claim 53, wherein the optical location indicator comprises an incandescent bulb.

60. The dissection tool of claim 53, wherein the optical location indicator comprises a color filter.

61. The dissection tool of claim 53, further comprising a fluid channel system extending between the proximal and distal ends of the dissecting member, the fluid channel system terminating in a port system.

62. The dissection tool of claim 61, wherein the fluid channel system is adapted to transport a pharmacological agent.

63. The dissection tool of claim 62, wherein the pharmacological agent comprises one or more of an analgesic, an antibiotic, and an antiseptic agent.
